(19) United States
(12) Patent Application Publication
(10) Pub. No.: US 2004/0166150 A1
(43) Pub. Date: Aug. 26, 2004

(54) Neutral-cationic lipid for systemic delivery of Factor VIII gene

(76) Inventors: Shi-Kun Huang, Castro Valley, CA (US); Bei Jin, Union City, CA (US); Weiming Zhang, San Francisco, CA (US); Yolanda Quinn, Daly City, CA (US); Samuel Zalipsky, Redwood City, CA (US)

Correspondence Address: ALZA Corporation, 1900 Charleston Road, P.O. Box 7210, M10-3, Mountain View, CA, 94039-7210, US

(21) Appl. No.: 10/786,747

(22) Filed: Feb. 25, 2004

Related U.S. Application Data
(63) Continuation of application No. 10/161,420, filed on May 28, 2002, now abandoned.
(63) Continuation-in-part of application No. 09/685,940, filed on Oct. 10, 2000.
(60) Provisional application No. 60/294,011, filed on May 29, 2001.
(60) Provisional application No. 60/158,693, filed on Oct. 8, 1999.

(51) Int. Cl. A61K 9/127

(52) U.S. Cl. 424/450

(57) ABSTRACT

A composition for delivery of Factor VIII is described. The composition is comprised of an expression vector encoding for Factor VIII and a lipid represented by the formula:

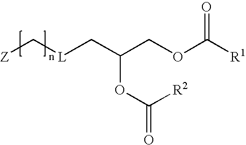

where each of R¹ and R² is an alkyl or alkenyl chain having between 8-24 carbon atoms; n=0-20, preferably 1-20; L is selected from the group consisting of (i) —X—(C═O)—Y—, (ii) —X—(C═O)—, and (iii) —X—, where X and Y are independently selected from oxygen, NH and a direct bond; and Z is a weakly basic moiety that has a pK of less than 7.4 and greater than about 4.0.

The application is a continuation-in-part of U.S. application Ser. No. 10/161,420 filed May 28, 2002, now pending, which claims the benefit of U.S. Application No. 60/294,011 filed May 29, 2001, now abandoned; and is also a continuation-in-part of U.S. application Ser. No. 09/685,940 filed Oct. 10, 2000, now pending, which claims the benefit of U.S. Application No. 60/158,693 filed Oct. 8, 1999, now abandoned, all of which are incorporated herein by reference in their entirety.

### FIELD OF THE INVENTION

The present invention relates to a composition for delivery of Factor VIII. More particularly, the invention relates to a composition comprising Factor VIII and a lipid having a moiety that is responsive to pH such that the lipid tends to be neutral at physiologic pH and is predominantly positively charged at a pH lower than physiologic pH.

### BACKGROUND OF THE INVENTION

The transmission of biologically active materials to cells is an essential component of a wide range of therapies. Such therapies include supplying a cell with a protein having a necessary biological activity, providing a new DNA molecule to a cell (gene therapy), immunizing a subject against a foreign protein (vaccination), immunizing a subject against a foreign protein by introducing a gene which encodes for the protein (gene vaccination) and inhibiting the production of a protein in a cell by providing the cell with a nucleic acid molecule which is antisense to mRNA encoding the protein or otherwise interfering with the mRNA encoding the protein.

There are, however, several obstacles to delivery of such agents to a cell, including the fact that the phospholipid bilayer comprising the outer membrane of most cells prohibits indiscriminate entry of materials into the cell. Approaches described to introduce active agents into cells include microinjection and electroporation. Other approaches involve viral vectors and chemical-mediated introduction.

Another approach for delivery of active agents to cells which has been described in the art is liposome-based delivery. In particular, delivery of genetic material to cells using liposomal carriers has been widely studied. It is generally understood that liposome vesicles are taken up by cells via endocytosis and enter the lysosomal degradation pathway. Thus, some effort towards designing liposomes that avoid degradation has been made. One approach has been to include in the liposome a pH sensitive lipid, such as palmitoylhomocysteine (Connor, J. et al., Proc. Natl. Acad. Sci. USA 81:1715 (1984); Chu, C.-J. and Szoka, F., J. Liposome Res. 4(1):361 (1994)). Such pH sensitive lipids at neutral pH are negatively charged and are stably incorporated into the liposome lipid bilayers. However, at weakly acidic pH (pH<6.8) the lipid becomes neutral in charge and changes in structure sufficiently to destabilize the liposome bilayers. The lipid when incorporated into a liposome that has been taken into an endosome, where the pH is reported to be between 5.0-6.0, destabilizes and causes a release of the liposome contents.

The use of cationic lipids, e.g., derivatives of glycolipids with a positively charged ammonium or sulfonium ion-containing headgroup, for delivery of negatively-charged biomolecules, such as oligonucleotides and gene fragments, as a liposome lipid bilayer component is also widely reported. The positively-charged headgroup of the lipid interacts with the negatively-charged cell surface, facilitating contact and delivery of the biomolecule to the cell.

Despite these efforts, delivery of biomolecules, such as oligonucleotides and other genetic material, to cells is still in need of improvement for improving transfer of agent to cells.

Factor VIII is one such biomolecule. Factor VIII is a protein normally present in plasma, and a decreased level or absence of the protein is the cause of hemophilia A. Hemophilia is an inherited disease now known to be present in different forms: hemophilia A, hemophilia B, and hemophilia C. Hemophilia A is the most frequent form with a clinical manifestation of a strong bleeding tendency. This is due to a lack of sufficient fibrin formation required for platelet plug stabilization, resulting in a plug which is easily dislodged with subsequent rebleeding at the injury site.

Therapeutic Factor VIII concentrates were traditionally prepared by fractionation of plasma. Today, Factor VIII is produced in cell culture using recombinent DNA techniques (Gitschier et al., Nature, 312:330(1984); EP-A-160457)). Factor VIII concentrates derived from human plasma contain several fragmented fully active Factor VIII forms (Andersson et al., Proc. Natl. Acad. Sci. USA, 83:2979 (1986)). The smallest active form has a molecular mass of 170 kDa and consists of two chains of 90 kDa and 80 kDa held together by a metal ion bridge. A recombinant Factor VIII product corresponding to this 170 kDa plasma Factor VII form is produced in cell culture.

While administration of the Factor VIII protein itself is effective for treatment of hemophilia, gene therapy for a long term, stable therapy is desired. Thus, delivery of DNA isolates as well as DNA expression vehicles containing gene sequences encoding human Factor VIII in expressible form to the cytoplasm of a cell is desired.

### SUMMARY OF THE INVENTION

Accordingly, it is an object of the invention to provide a composition for delivery of DNA expression vehicle encoding Factor VIII to a cell.

It is another object of the invention to provide a lipid for use in a composition for delivery of such a vehicle to a cell.

In one aspect, the invention includes a composition comprising a lipid represented by the formula:

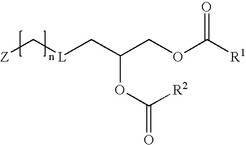

where each of R¹ and R² is an alkyl or alkenyl chain having between 8-24 carbon atoms; n=0-20, preferably n=1-20; L is selected from the group consisting of (i) —X—(C═O)—Y—, (ii) —X—(C═O)—, and (iii) —X—, where X and Y are independently selected from oxygen, NH and a direct bond; and Z is a weakly basic moiety that has a pK of less than 7.4 and greater than about 4.0. The composition also comprises a DNA expression vector encoding human Factor VIII.

In specific embodiments, X is NH and Y is oxygen. In other embodiments, L is a carbamate linkage (NH—(C═O)—O—), an ester linkage or a carbonate linkage. In a preferred embodiment, Z is an imidazole. Preferably, R¹ and R² is an unbranched alkyl or alkenyl chain having between 8-24 carbon atoms, and in a preferred embodiment, R¹ and R² are each stearyl groups (C₁₇H₃₅). In another preferred embodiment, n is between 1-10.

The liposomes, in one embodiment, include between 1-80 mole percent of the lipid having the formula shown above.

In another embodiment, Z is a moiety having a pK value between 5.0-6.5.

The composition, in another embodiment, includes a ligand for targeting. Preferably the ligand has binding affinity for a selected cell and is internalized by such cells.

In another embodiment, the composition includes between 5-20 mole percent of a vesicle-forming lipid derivatized with a hydrophilic polymer chain. The hydrophilic polymer chain in a preferred embodiment is polyethyleneglycol.

These and other objects and features of the invention will be more fully appreciated when the following detailed description of the invention is read in conjunction with the accompanying drawings.

### BRIEF DESCRIPTION OF THE DRAWINGS

FIG. 1 shows a synthetic scheme for the preparation of a lipid in accordance with the invention having a carbamante linkage and an imidazole Z group;

FIGS. 2A-2D show synthetic reaction schemes for preparation of pH responsive lipids in accord with the invention;

FIGS. 3A-3D show various structures of pH responsive lipids in accord with the invention;

FIG. 4 is a schematic illustration of preparation of targeted liposomes containing an expression vector encoding for Factor VIII and binding and internalization of the liposome to a target cell;

FIG. 5A is a graph showing the zeta potential, in mV, as a function of media pH for liposomes prepared with the lipid of the invention (open triangles), a cationic lipid (closed diamonds) and a neutral lipid (closed squares);

FIG. 5B is a graph showing the zeta potential, in mV, as a function of media pH for liposomes prepared with the neutral-cationic lipid of the invention (open diamonds), a liposome prepared with the neutral-cationic lipid and containing an entrapped DNA plasmid (closed square), a liposome prepared with the neutral-cationic lipid and with 10 mole percent PEG and containing an containing an entrapped DNA plasmid (triangles), and a liposome prepared with the lipid dimethyldioctadecylammonium (DDAB) and containing 10 mole percent PEG and an entrapped DNA plasmid (x);

FIG. 6 is a micrograph of gel electrophoresis assay of liposomes prepared with the pH-responsive lipid of the invention having entrapped DNA, where the liposomes were exposed to DNase I for 30 minutes (Lane 1); liposomes prepared with the pH-responsive lipid of the invention having entrapped DNA (Lane 2); DNA exposed to Dnase I for 30 minutes (Lane 3); DNA (Lane 4); and 1 kB DNA ladder standard (Lane 5);

FIGS. 7A-7D are images of micrographs of human lung tumor cells in vitro after transfection with liposomes prepared with the pH-responsive lipid of the invention and a targeting antibody. The liposomes include an entrapped plasmid encoding for green fluorescence protein, where FIG. 7A shows the transfected cells viewed under fluorescence microscopy and FIG. 7B shows the transfected cells viewed under light microscopy. FIGS. 7C-7D are micrographs of the cells after transfection with similar liposomes which do not have the targeting antibody, where the cells after incubation with the liposomes are shown under fluorescence microscopy in FIG. 7C and under light microscopy in FIG. 7D;

FIG. 8 is a bar graph showing the percentage of injected dose 30 minutes after intravenous injection in mice of liposomes composed of the cationic lipid DDAB; DDAB plus 10 mole percent PEG; imidazole-carbamate-distearoyl lipid with no PEG and with 4 mole percent and 10 mole percent PEG;

FIG. 9 is a bar graph showing the tissue distribution in mice of liposomes containing an entrapped DNA plasmid, where the liposomes contain either no PEG, 4 mole percent, or 10 mole percent PEG;

FIG. 10 is a graph showing the percentage of mouse body weight against number of days after treatment with saline (diamonds), liposomes prepared with the imidazole-carbamate-distearoyl lipid and having 150 μg DNA (squares) or 200 μg DNA (triangles); and liposomes prepared with DDAB and 100 μg DNA;

FIG. 11 is a bar graph showing the amount of Factor VIII in plasma, in ng/mL, 24 hours after intravenous injection in mice of 1 ml naked DNA, 0.2 mL naked DNA, 100 μg DNA in liposomes comprised of DDAM/cholesterol/PEG/galactose and imidazole-carbamate-distearoyl lipid/PHSPC/PEG/galactose;

FIG. 12 is a bar graph showing the amount of Factor VIII in mouse plasma, in ng/mL, 24 hours after intravenous injection of liposome/DNA complexes prepared from 100 μg DNA in imidazole-carbamate-distearoyl lipid/PHSPC, imidazole-carbamate-distearoyl lipid/PHSPC/PEG, and imidazole-carbamate-distearoyl lipid/PHSPC/PEG/galactose; and

FIG. 13 shows the retention of Factor VIII expression in the blood 24 hours and 1 week after intravenous injection into mice, where the amount of Factor VIII in plasma, in ng/mL, is shown for liposome/DNA complexes composed of imidazole-carbamate-distearoyl lipid/PHSPC/PEG/galactose.

### DETAILED DESCRIPTION OF THE INVENTION

I. Definitions

The terms below have the following meanings unless indicated otherwise.

As used herein, a “neutral” lipid is one that is uncharged, having no ionic character.

A “charged” lipid is one having a positive or negative charge, having ionic character.

“Vesicle-forming lipids” refers to amphipathic lipids which have hydrophobic and polar head group moieties, and which can form spontaneously into bilayer vesicles in water, as exemplified by phospholipids, or are stably incorporated into lipid bilayers, with the hydrophobic moiety in contact with the interior, hydrophobic region of the bilayer membrane, and the polar head group moiety oriented toward the exterior, polar surface of the membrane. The vesicle-forming lipids of this type typically include one or two hydrophobic acyl hydrocarbon chains or a steroid group, and may contain a chemically reactive group, such as an amine, acid, ester, aldehyde or alcohol, at the polar head group. Included in this class are the phospholipids, such as phosphatidyl choline (PC), phosphatidyl ethanolamine (PE), phosphatidic acid (PA), phosphatidyl inositol (PI), and sphingomyelin (SM), where the two hydrocarbon chains are typically between about 14-22 carbon atoms in length, and have varying degrees of unsaturation. Also included within the scope of the term “vesicle-forming lipids” are glycolipids, such as cerebrosides and gangliosides, and sterols, such as cholesterol.

“Alkyl” refers to a fully saturated monovalent radical containing carbon and hydrogen, and which may be branched or a straight chain. Examples of alkyl groups are methyl, ethyl, n-butyl, t-butyl, n-heptyl, and isopropyl. “Lower alkyl” refers to an alkyl radical of one to six carbon atoms, as exemplified by methyl, ethyl, n-butyl, i-butyl, t-butyl, isoamyl, n-pentyl, and isopentyl.

“Alkenyl” refers to monovalent radical containing carbon and hydrogen, which may be branched or a straight chain, and which contains one or more double bonds.

“Factor VIII” refers to a functional protein capable, in vitro or in vivo, of correcting human Factor VIII deficiencies, characterized by, for example, hemophilia A. Degradation forms and allelic variations are also intended.

“Expression vector” intends vectors which are capable of expressing DNA sequences contained therein, where such sequences are operably linked to other sequences capable of affecting their expression. In general, expression vectors of utility in recombinant DNA techniques take the form of “plasmids” which refer to circular double-stranded DNA loops. However, the invention intends to include other forms of expression vectors which serve equivalent functions.

Abbreviations: PEG: polyethylene glycol; mPEG: methoxy-terminated polyethylene glycol; Chol: cholesterol; PC: phosphatidyl choline; PHPC: partially hydrogenated phosphatidyl choline; PHEPC: partially hydrogenated egg phosphatidyl choline; HSPC: hydrogenated soy phosphatidyl choline; DSPE: distearoyl phosphatidyl ethanolamine; APD: 1-amino-2,3-propanediol; DTPA: diethylenetetramine pentaacetic acid; Bn: benzyl.

II. Cationic-Neutral Lipid

In one aspect, the invention includes lipids represented by the structure shown below:

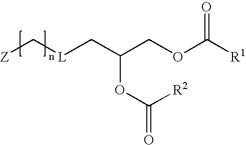

where each of R¹ and R² is an alkyl or alkenyl chain having between 8-24 carbon atoms; n=0-20, preferably n=1-20, and in a preferred embodiment is between 1-10; L is selected from the group consisting of (i) —X—(C═O)—Y—, (ii) —X—(C═O)—, and (iii) —X—, where X and Y are independently selected from oxygen, NH and a direct bond; and Z is a weakly basic moiety that has a pK of less than 7.4 and greater than about 4.0.

In another embodiment, Z is a moiety having a pK value between 4.5-7.0, more preferably between 5-6.5, and most preferably between 5-6.

The weakly basic moiety Z results in a lipid that at physiologic pH of 7.4 is predominantly, e.g., greater than 50%, neutral in charge but at a selected pH value less than physiologic pH tends to have a positive charge. By way of example, and in a preferred embodiment, Z is an imidazole moiety, which has a pK of about 6.0. At physiologic pH of 7.4, this moiety is predominantly neutral, but at pH values of less than 6.0, the moiety becomes predominantly positive. In support of the invention, a lipid having an imidazole moiety was prepared and used in preparation of liposomes, as will be discussed below.

In addition to imidazole, substituted imidazoles, as well as benzimidazoles and naphthimidazoles, can be used as the Z moiety in the structure given above, as long as the substitution does not alter the pKa to a value outside the desired range. Suitable substituents typically include alkyl, hydroxyalkyl, alkoxy, aryl, halogen, haloalkyl, amino, and aminoalkyl. Examples of such compounds reported to have pK's in the range of 5.0 to 6.0 include, but are not limited to, various methyl-substituted imidazoles and benzimidazoles, histamine, naphth[1,2-d]imidazole, 1H-naphth[2,3-d]imidazole, 2-phenylimidazole, 2-benzyl benzimidazole, 2,4-diphenyl-1H-imidazole, 4,5-diphenyl-1H-imidazole, 3-methyl-4(5)-chloro-1H-imidazole, 5(6)-fluoro-1H-benzimidazole, and 5-chloro-2-methyl-1H-benzimidazole.

Other nitrogen-containing heteroaromatics, such as pyridines, quinolines, isoquinolines, pyrimidines, phenanthrolines, and pyrazoles, can also be used as the Z group. Again, many such compounds having substituents selected from alkyl, hydroxyalkyl, alkoxy, aryl, halo, alkyl, amino, aminoalkyl, and hydroxy are reported to have pK's in the desired range. These include, among pyridines, 2-benzylpyridine, various methyl- and dimethylpyridines, as well as other lower alkyl and hydroxylalkyl pyridines, 3-aminopyridine, 4-(4-aminophenyl)pyridine, 2-(2-methoxyethyl)pyridine, 2-(4-aminophenyl)pyridine, 2-amino-4-chloropyridine, 4-(3-furanyl)pyridine, 4-vinylpyridine, and 4,4′-diamino-2,2′-bipyridine, all of which have reported pK's between 5.0 and 6.0. Quinolinoid compounds reported to have pK's in the desired range include, but are not limited to,

3-, 4-, 5-, 6-, 7- and 8-amino isoquinoline, various lower alkyl- and hydroxy-substituted quinolines and isoquinolines, 4-, 5-, 7- and 8-isoquinolinol, 5-, 6-, 7- and 8-quinolinol, 8-hydrazinoquinoline, 2-amino-4-methylquinazoline, 1,2,3,4-tetrahydro-8-quinolinol, 1,3-isoquinolinediamine, 2,4-quinolinediol, 5-amino-8-hydroxyquinoline, and quinuclidine. Also having pK's in the desired range are several amine-substituted pyrimidines, such as 4-(N,N-dimethylamino) pyrimidine, 4-(N-methylamino) pyrimidine, 4,5-pyrimidine diamine, 2-amino-4-methoxy pyrimidine, 2,4-diamino-5-chloropyrimidine, 4-amino-6-methylpyrimidine, 4-amino pyrimidine, and 4,6-pyrimidinediamine, as well as 4,6-pyrimidinediol. Various phenanthrolines, such as 1,10-, 1,8-, 1,9-, 2,8-, 2,9- and 3,7-phenanthroline, have pK's in the desired range, as do most of their lower alkyl-, hydroxyl-, and aryl-subsituted derivatives. Pyrazoles which may be used include, but are not limited to, 4,5-dihydro-1H-pyrazole, 4,5-dihydro-4-methyl-3H-pyrazole, 1-hydroxy-1H-pyrazole, and 4-aminopyrazole.

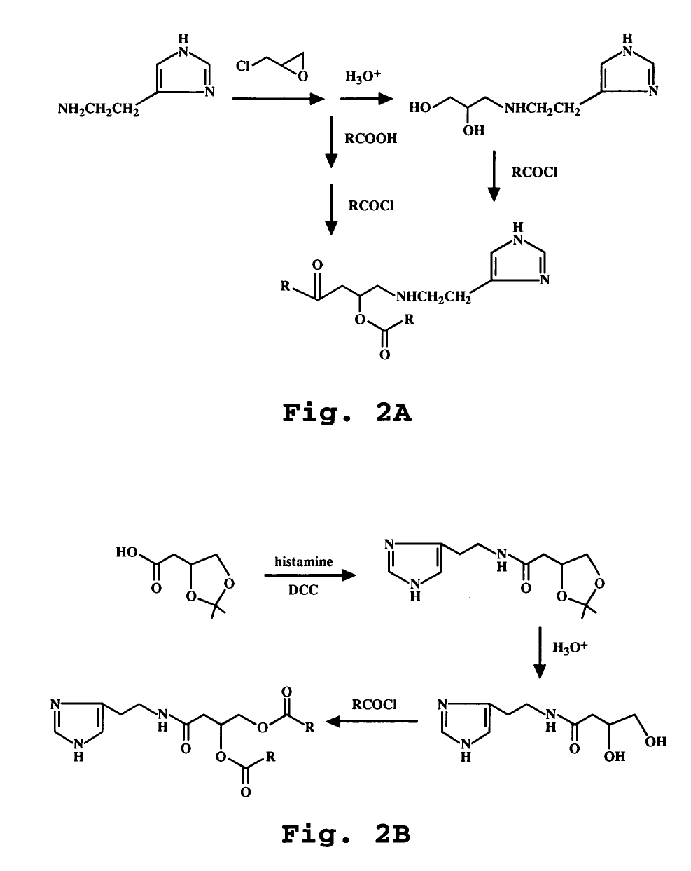

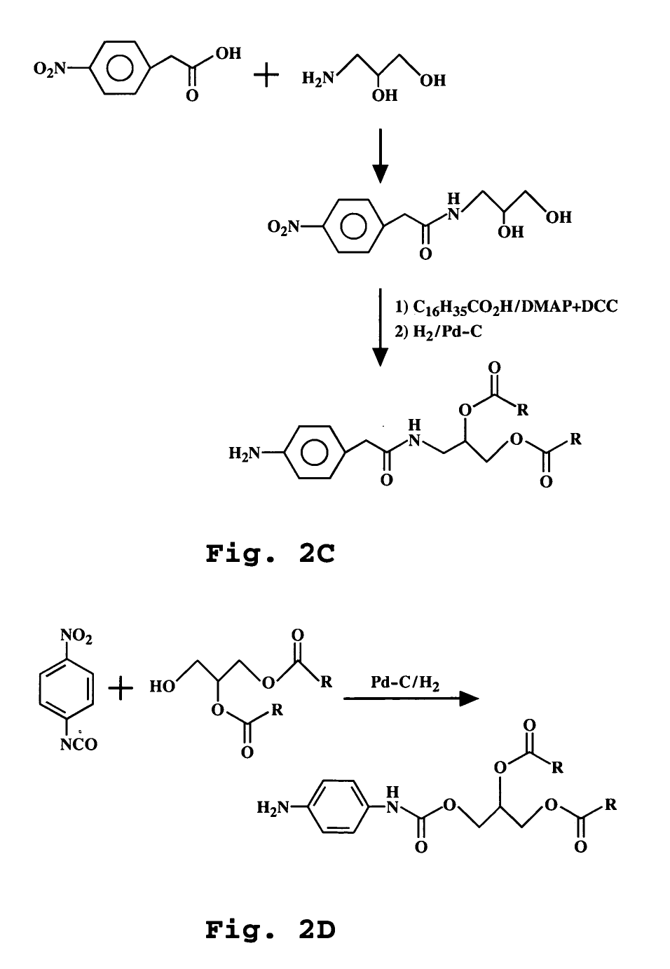

Many nitrogen-substituted aromatics, such as anilines (as shown in FIGS. 2C-D and 3A-B) and naphthylamines, are also suitable embodiments of the group Z. Anilines and naphthylamines further substituted with groups selected from methyl or other lower alkyl, hydroxyalkyl, alkoxy, hydroxyl, additional amine groups, aminoalkyl, halogen, and haloalkyl are generally reported to have pKa's in the desired range. Other amine-substituted aromatics which can be used include 2-aminophenazine, 2,3-pyrazinediamine, 4- and 5-aminoacenaphthene, 3- and 4-amino pyridazine, 2-amino-4-methylquinazoline, 5-aminoindane, 5-aminoindazole, 3,3′,4,4′-biphenyl tetramine, and 1,2- and 2,3-diaminoanthraquinone.

Also included as embodiments of Z are certain acyclic amine compounds, such as N,N′-dimethylguanidine and various substituted hydrazines, such as trimethylhydrazine, tetramethylhydrazine, 1-methyl-1-phenylhydrazine, 1-naphthalenylhydrazine, and 2-, 3-, and 4-methylphenyl hydrazine, all of which are reported to have pKa′ between 4.5 and 7.0. Alicyclic compounds having pKa's in this range include 1-pyrrolidineethanamine, 1-piperidineethanamine, hexamethylenetetramine, and 1,5-diazabicyclo[3.3.3]undecane.

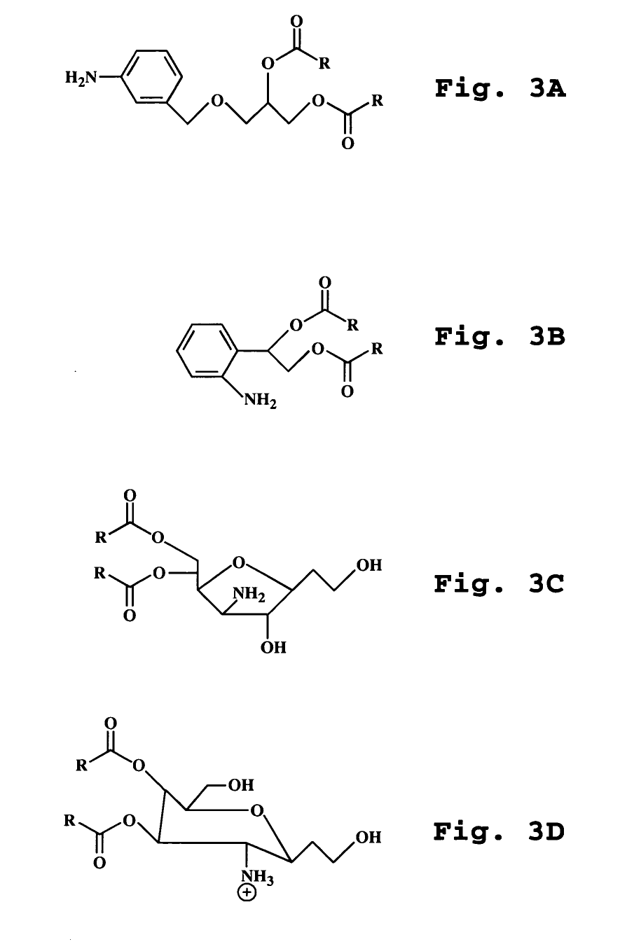

Also suitable as the Z moiety in the structure given above are certain aminosugars, such as shown in FIGS. 3C-D.

The above listings give examples of compounds having pKa's between 4.5 and 7.0 which may be used as pH-responsive groups in the lipid conjugates of the invention; these listings are not intended to be limiting. In selected embodiments, the group Z is a imidazole, aniline, aminosugar or derivative thereof. Preferably, the effective pKa of the group Z is not significantly affected by its attachment to the lipid group. Examples of linked conjugates are given below.

The lipids of the invention include a neutral linkage L joining the Z moiety and the tail portion of the lipid. Linkage L is variable, and in preferred embodiments is selected from a carbamate, an ester, an amide, a carbonate, a urea, an amine, and an ether. In a preferred lipid prepared in support of the invention, a carbamate linkage, where L is —X—(C═O)—Y—, X being NH and Y being oxygen, was prepared.

In the tail portion of the lipid, R¹ and R² are the same or different and can be an unbranched alkyl or alkenyl chain having between 8-24 carbon atoms. More preferably, the R¹ and R² groups are between 12-22 carbon atoms in length, with R¹ R²═C₁₇H₃₅ (such that the group is a stearyl group) or R¹═R²=C₁₇H₃₃ (such that the group is an oleoyl group).

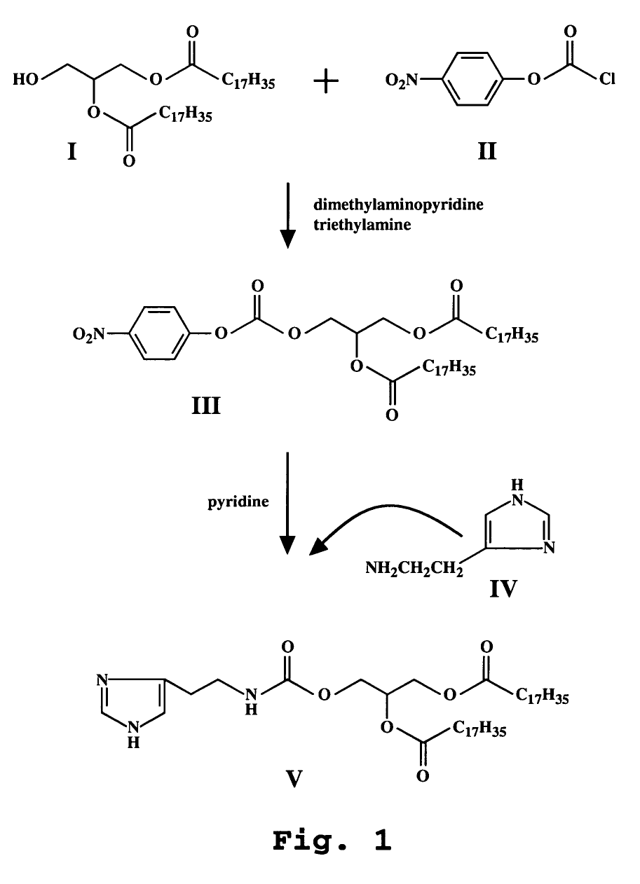

The lipid of the invention can be prepared using standard synthetic methods. As mentioned above, in studies performed in support of the invention, a lipid having the structure shown above, where Z is an imidazole, n=2, L is a carbamate and R¹═R²=C₁₇H₃₅, was prepared. A reaction scheme for preparation of the exemplary lipid is illustrated in FIG. 1 and details of the synthesis are provided in Example 1. Briefly, the para-nitrophenyl carbonate of 1,2-distearoyl glycerol (Compound II) was prepared from 1,2-distearoyl-sn-glycerol (Compound I) and para-nitrophenyl chloroformate (Compound II) and reacted with histamine (Compound IV) to yield a lipid (Compound V) having a imidazole moiety linked to a distearoyl tail via a carbamate linkage. A similar route, using glycerol in place of 1-amino-2,3-propanediol, can be used to produce a carbonate-linked product (L=—O—(C═O)—O—CH₂—).

Preparation of the lipid having other linkages is readily done by those of skill in the art using conventional methods. Other linkages include ether (L=—O—CH₂—) and ester linkages (L=—O—(C═O)—), as well as urea amide, and amine linkages (i.e., where L=—NH—(C═O)—NH—, —NH—(C═O)— or —NH—). A keto linkage, where X is a direct bond, is also possible. FIGS. 2A-2B illustrate preparation of an amine-linked lipid (FIG. 2A) and a lipid having an NH— containing linkage (FIG. 2B). In FIG. 2A, the terminal amine of histamine is reacted with with glycidyl chloride, the resulting epoxide is hydrolyzed, and the resulting diol is acylated.

In FIG. 2B, a lipid having an NH— containing linkage (L=—O—(C═O)— or —O—(C═O)—CH₂—) is prepared, for example, by reacting histamine with an activated derivative of glyceric acid acetonide (2,2-dimethyl-1,3-dioxolane-4-carboxylic acid) or the four-carbon homolog, 2,2-dimethyl-1,3-dioxolane-4-acetic acid, as shown. The diol is then deprotected and acylated.

FIGS. 2C and 2D show other reaction schemes for preparation of pH-responsive lipids in accord with the invention. In FIG. 2C, 4-nitrobenzoic acid is condensed with 1-amino-2,3-propanediol, giving an amide linkage; the diol is acylated and the nitro group reduced to an amine to give the the product, a lipid-aniline conjugate. In FIG. 2D, the initial condensation reaction is between an alcohol (diacylgycerol) and an isocyanate, giving a carbamate linkage in the product.

FIGS. 3A-3D show various structures of pH-responsive lipids in accord with the invention, where FIGS. 3A-3B show further lipids having an aromatic amine as the “Z” moiety, and FIGS. 3C-3D show lipids having an aminosugar attached to a lipid. Synthesis of these lipids can be readily performed by those of skill in the art using commercially available starting materials. For example, the product of FIG. 3A may be prepared by reaction of m-nitrobenzyl bromide and a diacylglycerol, giving the ether linkage, followed by reduction of the nitro group. The product of FIG. 3B is prepared from commercially available (2-nitrophenyl)-1,2-ethanediol by acylation of the diol and reduction of the nitro group. To prepare the aminosugar-lipid conjugate shown in FIG. 3C, D-glucose (furanose form) is protected by reaction with two molecules of acetone, and the free hydroxyl group is sequentially reacted with TsCl, sodium azide, and iodine to give an intermediate nitro compound. The exocyclic diol is deprotected and acylated, and the nitro group reduced to the amine. The compound of FIG. 3D can be prepared in a similar manner from D-galactose.

III. Liposome Composition

A. Liposome Components

Liposomes containing the lipid described above can be prepared by a variety of techniques, such as those detailed in Szoka, F., Jr., et al., Ann. Rev. Biophys. Bioeng. 9:467 (1980), and specific examples of liposomes prepared in support of the present invention will be described below. Typically, the liposomes are multilamellar vesicles (MLVs), which can be formed by simple lipid-film hydration techniques. In this procedure, a mixture of liposome-forming lipids of the type detailed below are dissolved in a suitable organic solvent which is then evaporated in a vessel to form a thin film. The lipid film is then covered by an aqueous medium, hydrating to form MLVs, typically with sizes between about 0.1 to 10 microns.

Liposomes prepared in accordance with the invention include between about 1-80 mole percent of the lipid having the structure given above. In preferred embodiments, liposomes include between 5-50 mole percent of the lipid. The remainder of the liposome lipid components can include a variety of vesicle-forming lipids—lipids that can form spontaneously into bilayer vesicles in water, as exemplified by the phospholipids. The vesicle-forming lipids of this type are preferably ones having two hydrocarbon chains, typically acyl chains, and a head group, either polar or nonpolar. There are a variety of synthetic vesicle-forming lipids and naturally-occurring vesicle-forming lipids, including the phospholipids, such as phosphatidylcholine, phosphatidylethanolamine, phosphatidylglycerol, phosphatidylserine, phosphatidic acid, phosphatidylinositol, and sphingomyelin, where the two hydrocarbon chains are typically between about 12-22 carbon atoms in length, and have varying degrees of unsaturation.

The liposomes can also include a lipid that is stably incorporated into the liposome lipid bilayer, such as diacylglycerols, lyso-phospholipids, fatty acids, glycolipids, cerebrosides and sterols, such as cholesterol.

In one embodiment, the liposomes of the invention include a surface coating of a hydrophilic polymer chain. “Surface-coating” refers to the coating of any hydrophilic polymer on the surface of liposomes. The hydrophilic polymer is included in the liposome by including in the liposome composition one or more vesicle-forming lipids derivatized with a hydrophilic polymer chain. Liposomes having such a coating are well known in the art, and have been described, for example in U.S. Pat. No. 5,013,556. The surface coating of hydrophilic polymer chains is effective to increase the in vivo blood circulation lifetime of the liposomes when compared to liposomes lacking such a coating.

Vesicle-forming lipids suitable for derivatization with a hydrophilic polymer include any of those lipids listed above, and, in particular phospholipids, such as distearoyl phosphatidylethanolamine (DSPE).

Hydrophilic polymers suitable for derivatization with a vesicle-forming lipid include polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyaspartamide and hydrophilic peptide sequences. The polymers may be employed as homopolymers or as block or random copolymers.

A preferred hydrophilic polymer chain is polyethyleneglycol (PEG), preferably as a PEG chain having a molecular weight between 500-10,000 daltons, more preferably between 1,000-5,000 daltons. Methoxy or ethoxy-capped analogues of PEG are also preferred hydrophilic polymers, commercially available in a variety of polymer sizes, e.g., 120-20,000 daltons.

Preparation of vesicle-forming lipids derivatized with hydrophilic polymers has been described, for example in U.S. Pat. No. 5,395,619, which is expressly incorporated herein by reference, and by Zalipsky in STEALTH LIPOSOMES (D. Lasic and F. Martin, Eds., CRC Press, Chapter 9 (1995)).

Liposomes having such a coating are preferably prepared by including between 1-20 mole percent of the derivatized lipid with the remaining liposome forming components, e.g., vesicle-forming lipids. Exemplary methods of preparing derivatized lipids and of forming polymer-coated liposomes have been described in co-owned U.S. Pat. Nos. 5,013,556, 5,631,018 and 5,395,619, which are incorporated herein by reference. It will be appreciated that the hydrophilic polymer may be stably coupled to the lipid, or coupled through an unstable linkage which allows the coated liposomes to shed the coating of polymer chains as they circulate in the bloodstream or in response to a stimulus.

The liposomes also include an entrapped agent, where “entrapped” is intended to include encapsulation of an agent in the aqueous core and aqueous spaces of liposomes as well as entrapment of an agent in the lipid bilayer(s) of the liposomes.

Agents contemplated for use in the composition of the invention are widely varied, and include agents for therapeutic applications as well as diagnostic applications. The therapeutic or diagnostic agent of choice can be incorporated into liposomes by standard methods, including (i) passive entrapment of a water-soluble compound by hydrating a lipid film with an aqueous solution of the agent, (ii) passive entrapment of a lipophilic compound by hydrating a lipid film containing the agent, and (iii) loading an ionizable drug against an inside/outside liposome pH gradient. Other methods, such as reverse evaporation phase liposome preparation, are also suitable.

The entrapped agent in the present invention is an expression vector encoding for Factor VIII. It will be appreciated that other agents, such as pharmacological drugs or compounds, can also be simultaneously entrapped. Suitable vectors specific for Factor VIII and Factor VIII sequences are described in the art, for example, in U.S. Pat. No. 5,668,108. Administration of a vector encoding for Factor IX is also contemplated. It will be appreciated that the composition of the invention is suitable for delivery of a variety of DNA and RNA based nucleic acids known in the art, and coding sequences for specific genes of interest can be retrieved from DNA sequence databanks, such as GenBank or EMBL. For example, polynucleotides for treatment of viral, malignant and inflammatory diseases and conditions, such as, cystic fibrosis, adenosine deaminase deficiency and AIDS, have been described. Treatment of cancers by administration of tumor suppressor genes, such as APC, DPC4, NF-1, NF-2, MTS1, RB, p53, WT1, BRCA1, BRCA2 and VHL, are contemplated.

The nucleic acid in a preferred embodiment is inserted into a plasmid, preferably one that is a circularized or closed double-stranded molecule having sizes preferably in the 5-40 Kbp (kilo basepair) range. Such plasmids are constructed according to well-known methods and include a therapeutic gene, i.e., the gene to be expressed in gene therapy, under the control of suitable promoter and enhancer, and other elements necessary for replication within the host cell and/or integration into the host-cell genome. Methods for preparing plasmids useful for gene therapy are widely known and referenced.

Polynucleotides, oligonucleotides, other nucleic acids, such as a DNA plasmid, can be entrapped in the liposome by passive entrapment during hydration of the lipid film. Other procedures for entrapping polynucleotides include condensing the nucleic acid in single-molecule form, where the nucleic acid is suspended in an aqueous medium containing protamine sulfate, spermine, spermidine, histone, lysine, mixtures thereof, or other suitable polycationic condensing agent, under conditions effective to condense the nucleic acid into small particles. The solution of condensed nucleic acid molecules is used to rehydrate a dried lipid film to form liposomes with the condensed nucleic acid in entrapped form.

Liposomes prepared in accordance with the invention, can be sized to have substantially homogeneous sizes in a selected size range, typically between about 0.01 to 0.5 microns, more preferably between 0.03-0.40 microns. One effective sizing method for REVs and MLVs involves extruding an aqueous suspension of the liposomes through a series of polycarbonate membranes having a selected uniform pore size in the range of 0.03 to 0.2 micron, typically 0.05, 0.08, 0.1, or 0.2 microns. The pore size of the membrane corresponds roughly to the largest sizes of liposomes produced by extrusion through that membrane, particularly where the preparation is extruded two or more times through the same membrane. Homogenization methods are also useful for down-sizing liposomes to sizes of 100 nm or less (Martin, F. J., in SPECIALIZED DRUG DELIVERY SYSTEMS-MANUFACTURING AND PRODUCTION TECHNOLOGY, (P. Tyle, Ed.) Marcel Dekker, New York, pp. 267-316 (1990)).

1. Targeting Ligand

In yet another embodiment, the liposomes may be prepared to contain surface groups, such as antibodies or antibody fragments, small effector molecules for interacting with cell-surface receptors, antigens, and other like compounds for achieving desired target-binding properties to specific cell populations. Such ligands can be included in the liposomes by including in the liposomal lipids a lipid derivatized with the targeting molecule, or a lipid having a polar-head chemical group that can be derivatized with the targeting molecule in preformed liposomes.

Lipids can be derivatized with the targeting ligand by covalently attaching the ligand to the free distal end of a hydrophilic polymer chain, which is attached at its proximal end to a vesicle-forming lipid. There are a wide variety of techniques for attaching a selected hydrophilic polymer to a selected lipid and activating the free, unattached end of the polymer for reaction with a selected ligand, and in particular, the hydrophilic polymer polyethyleneglycol (PEG) has been widely studied (Allen, T. M., et al., Biochemicia et Biophysica Acta 1237:99-108 (1995); Zalipsky, S., Bioconjugate Chem., 4(4):296-299 (1993); Zalipsky, S., et al., FEBS Lett. 353:71-74 (1994); Zalipsky, S., et al., Bioconjugate Chemistry, 705-708 (1995); Zalipsky, S., in STEALTH LIPOSOMES (D. Lasic and F. Martin, Eds.) Chapter 9, CRC Press, Boca Raton, Fla. (1995)).

Targeting ligands are well known to those of skill in the art, and in a preferred embodiment of the present invention, the ligand is one that has binding affinity to hepatocytes and/or which initiates internalization of the liposomes by the cells. A preferred targeting ligand is galactose.

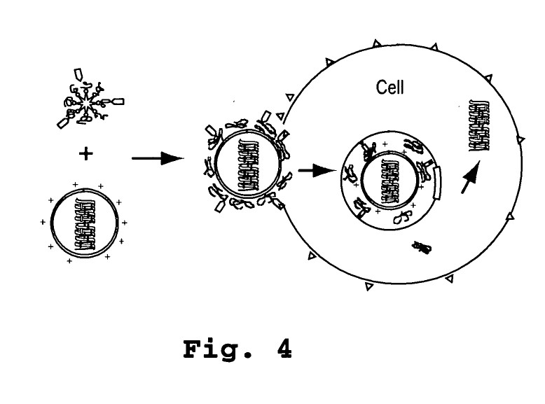

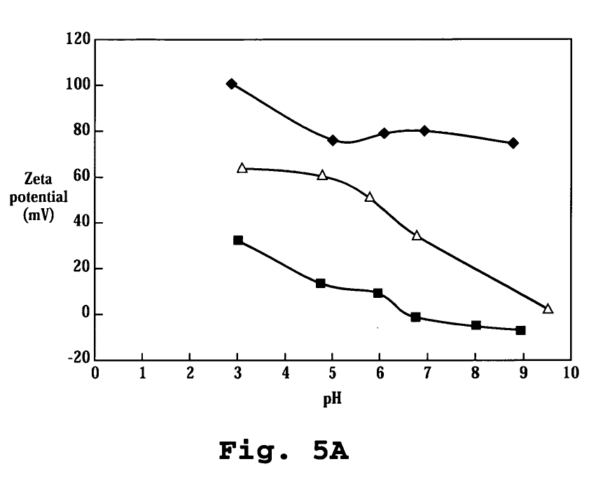

FIG. 4 is an illustration of another method of preparing a liposome composition with a targeting conjugate. Here, a liposome comprised of the neutral cationic lipid is prepared with the DNA expression vector entrapped in the liposome. Separately, a micellar composition of a lipid-derivatized hydrophilic polymer, such as PEG, having a targeting ligand covalently attached to the distal end of the PEG chain is prepared. The micellar composition is incubated with the liposomes under conditions to achieve insertion of the lipid-derivatized PEG micelles into the liposome lipid bilayer. Suitable incubation conditions to achieve insertion are described, for example, in U.S. Pat. No. 6,056,973, incorporated herein by reference in its entirety.

IV. Preparation and Characterization of Exemplary Compositions

Liposomes including the lipid described in Example 1, having an imidazole moiety linked to a distearoyl tail via a carbamate linkage were prepared as described in Example 2. The liposomes were composed of 60 mole percent partially hydrogenated soy-bean phosphatidylcholine (PHSPC) and 40 mole percent of the imidazole-carbamate-distearoyl lipid. The liposomes in the composition had an average particle size of 80 nm after sonication.

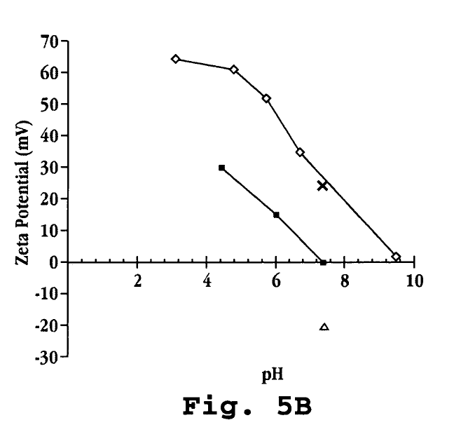

The zeta potential of these liposomes was measured as a function of pH, and the results are shown in FIG. 5A (open triangles). For comparison, two liposome compositions not containing the lipid of the invention were prepared, one composition including a cationic lipid the other composition consisting of a single neutral lipid, PHSPC. The cationic liposome composition was composed of 55 mole percent of dimethyldioctadecylammonium (DDAB) and 45 mole percent of cholesterol.

The zeta potential values provide a measure of the apparent charge on the outer surface of the liposomes. More specifically, the zeta potential is a measure of the potential that arises across the interface between a liquid boundary layer in contact with a solid and the movable diffuse layer in the body of the liquid, e.g., the slipping plane. Zeta potential values were measured as set forth in the methods section below, using a commercially available apparatus.

In FIG. 5A, the liposome prepared with the imidazole-carbamate-distearoyl lipid (open triangles) show a strong relationship between zeta potential and pH of the surrounding media. As seen, at pH values of less than about 5.0, the zeta potential is relatively constant at about 65 mV. As the pH of the media is increased, the zeta potential decreases rapidly. In contrast, cationic liposomes (e.g., liposomes of DDAB-cholesterol, solid diamonds) and the neutral liposome formulation (solid squares) had less change in zeta potential as the pH of the suspension media increased.

The rapid change in zeta potential of the liposomes composed with imidazole-carbamate-distearoyl lipids as the pH of the suspension media increased is due to the pK property of the imidazole moiety. The pK of imidazole is around pH 6. At pH less than 6, the imidazole moiety is predominately, e.g., greater than 50%, positive in charge, and the zeta potential of the imidazole-carbamate-distearoyl lipid-containing liposomes tens to positive. At pH higher than 6, imidazole turns to neutral charge, e.g., greater than 50% neutral, and the positive zeta potential of the imidazole-carbamate-distearoyl lipid-containing liposomes decreases, or tends to neutral.

FIG. 5B a graph showing the zeta potential, in mV, as a function of media pH for liposomes prepared with the neutral-cationic lipid of the invention and partially hydrogenated soybean phosphatidylcholine (PHSPC) in a 60:40 molar ratio (open diamonds). As seen, the surface potential of the complexes was 25 mV at pH 5 and 0 mV at pH 7.4. Also shown are the zeta potentials as a function of pH for the same liposome further including an entrapped DNA plasmid (closed square) and for the same liposome including an entrapped DNA plasmid plus 10 mole percent PEG (triangles). For comparison, the zeta potential at pH 7.4 for a liposome prepared with the lipid DDAB and containing 10 mole percent PEG and an entrapped DNA plasmid (x) is shown.

Electron micrographs (not shown here) of the liposomes having an entrapped DNA plasmid show the presence of a lipid bilayer at the outer surface. The DNA plasmid is enveloped by the lipid bilayer, but may have cationic lipids which may not be associated with the lipid bilayer. The term “complex” is used herein to refer to a DNA plasmid surrounded by a lipid bilayer but possibly having other lipids associated with the DNA plasmid.

In another study performed in support of the invention, liposomes including the imidazole-carbamate-distearoyl lipid (prepared as described in Example 1) with entrapped DNA were prepared. As described in Example 3A, condensed plasmid DNA was contacted with liposomes comprised of a 60/40 molar ratio of PHSPC and the imidazole-carbamate-distearoyl lipid. The pH of the liposome solution was adjusted to about 4.0 prior to contact with the condensed DNA. At this pH, the imidazole head group on the lipid is positively charged, so that the negatively charged DNA becomes electrostatically bound to the lipid. With continuous stirring, the liposomes form around the DNA, entrapping it within the lipid bilayer. Accordingly, the invention provides a method for efficiently entrapping a negatively-charged agent by preparing liposomes with a pH-responsive lipid and contacting the agent with the liposomes under conditions where the pH-responsive lipid tends toward positive charge.

The liposomes having the entrapped DNA and a sample of DNA alone were treated with DNase I for 30 minutes (see Example 3B). After the treatment period, an aliquot of each was loaded onto an agarose gel containing ethidium bromide and electrophoresed. Samples of the same liposomes and of DNA not treated with DNase I were also loaded onto the gel.

FIG. 6 is a micrograph of the gel electrophoresis assay of these samples, where Lane 1 is the DNase-treated liposomes, Lane 2 is the liposomes (not treated with DNase I); Lane 3 is the DNA treated with DNase I, Lane 4 is the DNA, and Lane 5 is a 1 kB DNA ladder standard.

FIG. 6 shows that the DNA entrapped in the liposomes was protected from digestion by the DNase I, as evidenced by comparing Lane 1 with Lane 3, where the DNA alone was digested by the DNase I.

In another study performed in support of the invention, liposomes including a pH responsive lipid and a targeting antibody were prepared. These liposomes were used for in vitro transfection of human lung tumor cells, as will now be described.

A DNA reporter plasmid vector pEGFP-C1 (Clontech, Palo Alto Calif.) containing green fluorescence protein gene was entrapped in liposomes according to the procedure of Example 3A. The ratio of total lipids to DNA in the liposomes was 14 nmole lipids per 1 μg DNA. After the DNA was entrapped in the liposomes, an anti-integrin antibody 1F11 Fab′ inserted into the lipid bilayer by incubating the liposomes with micelles of 1F11-Fab′-conjuated to polyethyleneglycol-DSPE. The 1F11-Fab′-PEG-DSPE conjugate was prepared using conventional technology by attaching the antibody at the N-terminal maleimide of PEG-DSPE, as has been described, for example, in Zalipsky in STEALTH LIPOSOMES (D. Lasic and F. Martin, Eds., CRC Press, Chapter 9 (1995). The antibody-containing micelles and the liposomes were incubated overnight at room temperature.

Human lung tumor cell line 2E9 having an integrin receptor were incubated with the liposomes in vitro. Liposomes containing the 1F11-PEG-DSPE conjugate and control liposomes with no antibody (containing PDG-DSPE absent the antibody) were incubated with the cells at a concentration of 5 μg DNA/70 nmole lipid per ml for 4 hours at 37^{∀}C. After the incubation period, the medium was changed to remove the liposomes.

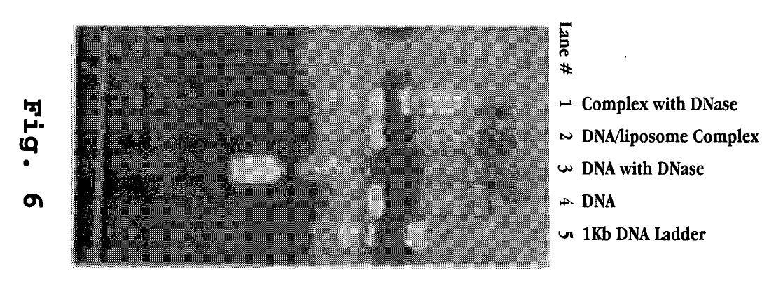

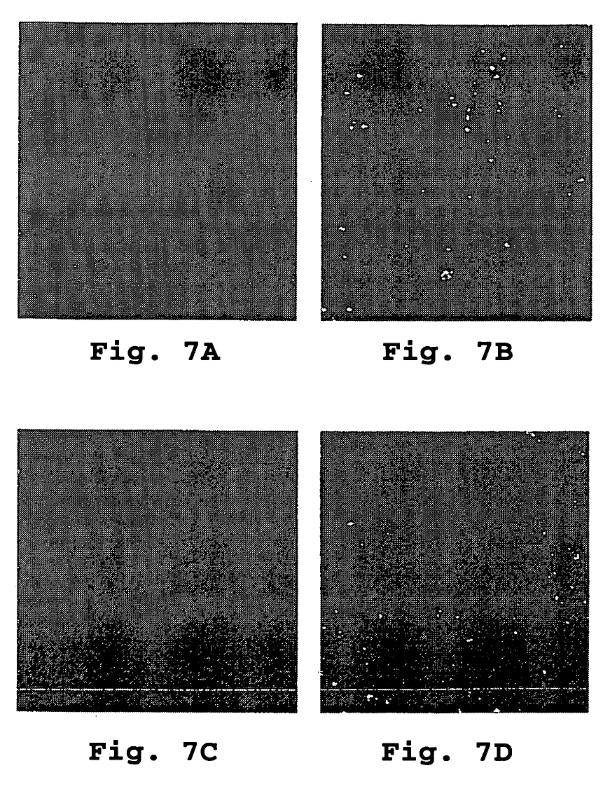

Green fluorescence was examined 24 hours after transfection and the results are shown in FIGS. 6A-6D. FIGS. 6A-6B are micrographs for the cells transfected with the 1F11-conjugated liposomes, where FIG. 7A shows the cells viewed under fluorescence microscopy and FIG. 7B shows the cells viewed under light microcopy. FIG. 7A clearly shows that the cells were transfected, as evidenced by the light regions which correspond to fluorescing cells. FIGS. 7C-7D are images of the cells transfected with the control formulation containing no targeting antibody. No transfection occurred, as evidenced by the lack of fluorescence when the cells are viewed by fluorescence microscopy (FIG. 7C).

V. In Vivo Administration of Exemplary Composition

In other studies conducted in support of the invention, a liposome composition including a plasmid encoding for Factor VIII was prepared and administered intravenously to mice, as will now be described. In these studies, a DNA plasmid encoding human Factor VIII gene with the B-domain deleted.

Complexes were prepared with liposomes comprised of the neutral cationic lipid described above (imidazole-carbamate-distearoyl lipid) and partially hydrogenated soybean phosphatidylcholine, in a 60:40 molar ratio. The surface of the complexes was modified with 10 mole percent mPEG-DSPE (PEG MW 2000 D) and approximately 20 galactose-PEG-distearoylphosphatidic acid molecules per complex.

Mice (C57BL6) were divided into 5 treatment groups with 3 mice per group. The treatment groups are shown in Table 1.

TABLE 1

| | | | DNA | | injection |
|---|---|---|---|---|---|
| | | | (μg)/Lipid | DNA Conc. | volume |
| Group No. | Lipid Composition¹ | Lipid Ratio | (nmol) Ratio | (μg/mL) | (mL) |
| 1: Control | n/a | n/a | n/a | 30 | 1 |
| 2 | NCL/PHSPC | 40:60 | 1:30 | 250 | 0.4 |
| 3 | NCL/PHSPC/PEG | 40:60:10 | 1:30 | 250 | 0.4 |
| 4 | NCL/PHSPC/Gal- | 40:60:10 | 1:30 | 250 | 0.4 |
| | PEG | | | | |
| 5 | NCL/PHSPC | 40:60 | 1:14 | 250 | 0.4 |

¹NCL = neutral cationic lipid (imidazole-carbamate-distearoyl lipid);
PHSPC = partially hydrogenated soy phosphatidylchloline;
Gal = galactose;
PEG = polyethyleneglycol

The control group no. 1 was injected intravenously with free DNA pAPC-10. All other test groups were injected intravenously with the indicated DNA/liposome formulation with or without a galactose targeting ligand. The mice were anesthetized with isofluorane 24 hours after injection. 200 μL of blood was collected from retroorbital bleeding using heparinized micro-hematocrit capillary tubes.

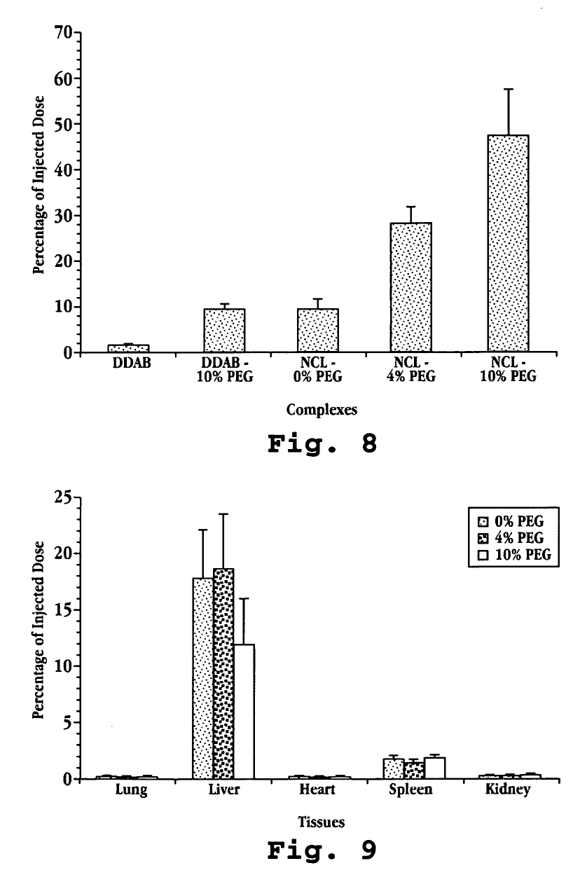

FIG. 8 is a bar graph showing the percentage of injected dose 30 minutes after intravenous injection of liposomes composed of the cationic lipid DDAB; DDAB plus 10 mole percent PEG; imidazole-carbamate-distearoyl lipid with no PEG and with 4 mole percent and 10 mole percent PEG. The percentage of injected dose was measured by the fluorescence intensity level of rhodamine-labeled liposomes in the plasma sample. The liposomes prepared with the neutral-cationic lipid imidazole-carbamate-distearoyl and 10 mole perecent PEG had about 47% of the dose in circulation 30 minutes after injection. In contrast, the liposomes prepared with the cationic lipid DDAB, with and without PEG, had 10% or less in circulation 30 minutes after administration.

FIG. 9 shows the tissue distribution of liposomes containing an entrapped DNA plasmid, where the liposomes were prepared with the imidazole-carbamate-distearoyl lipid and with either no PEG, 4 mole percent, or 10 mole percent PEG. As seen, distribution in the liver was this highest, with between 12-18% of the injected dose in the liver 30 minutes after injection. This result is in contrast to the distribution typically observed after administration of cationic liposome/DNA complexes where localization in the lung is strong due to the positive surface charge of the complexes. While a positive charge is important for DNA complexation, cell interaction, and DNA transfer from the lysosomes to the cytoplasm after internalization, a positive charge is undesirable for biodistribution. Here, the absence of surface charge at physiologic pH leads to a distribution in the liver 10 fold higher than the lung.

In another study, the in vivo toxicity of the neutral-cationic lipid imidazole-carbamate-distearoyl was studied. Mice were treated with liposomes prepared with the complex and the body weight of the mice was monitored for 6 days. FIG. 10 shows the results as the percentage of body weight against number of days after treatment. Mice treated with saline (diamonds) or with liposomes prepared with the imidazole-carbamate-distearoyl lipid and having 150 μg DNA (squares) or 200 μg DNA (triangles) have a similar profile. For comparison, liposomes prepared with DDAB and 100 μg DNA were also administered to mice (x symbols), and a drop in body weight was observed from days 1-3.

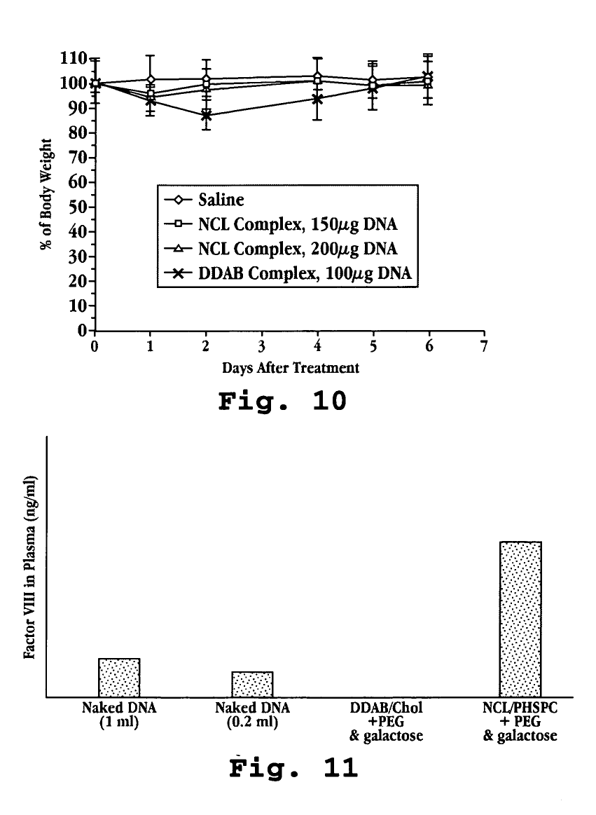

FIG. 11 is a bar graph showing the amount of Factor VIII in plasma, in ng/mL, 24 hours after intravenous injection of 100 μg in 1 ml naked DNA encoding for Factor VIII, 100 μg in 0.2 mL naked DNA encoding for Factor VIII, 100 μg DNA encoding for Factor VIII entrapped in liposomes comprised of DDAB/cholesterol/PEG/galactose and imidazole-carbamate-distearoyl lipid/PHSPC/PEG/galactose. The composition including the lipid of the invention achieved the highest expression of Factor VIII in the plasma. The mean plasma concentration of Factor VIII following administration from the liposome complexes prepared with the imidazole-carbamate-distearoyl lipid was 4.7 (±2.8) ng/mL. Treatment with complexes lacking PEG and ligand surface targeting moieties yielded an average plasma level of 0.86 (±0.3) ng/mL. No Factor VIII expression was detected with the conventional DDAB cationic liposome/DNA complexes under the same conditions. Administration of 100 μg of naked plasmid DNA by 1 mL bolus injection volume and 0.2 mL small injection volume achieved plasma concentrations of Factor VIII of 1.2 (±0.3) ng/mL and 0.80 (±1.3) ng/mL, respectively.

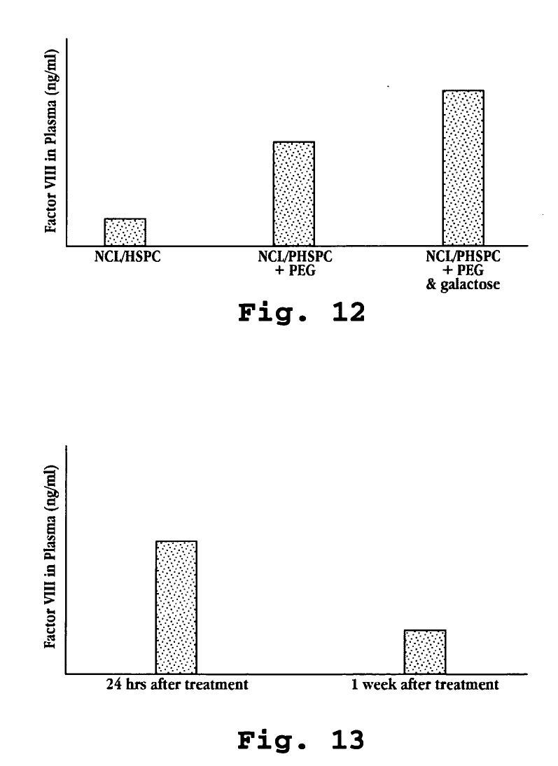

FIG. 12 shows the amount of Factor VIII in plasma, in ng/mL, 24 hours after intravenous injection of liposome/DNA complexes prepared from 100 μg DNA encoding for Factor VIII and imidazole-carbamate-distearoyl lipid/PHSPC, imidazole-carbamate-distearoyl lipid/PHSPC/PEG, or imidazole-carbamate-distearoyl lipid/PHSPC/PEG/galactose. The liposome composition with the galactose targeting ligand achieved the highest expression of Factor VIII in the plasma.

FIG. 13 shows the retention of Factor VIII expression in the blood 24 hours and 1 week after intravenous injection into mice, where the amount of Factor VIII in plasma, in ng/mL, is shown for liposome/DNA complexes composed of imidazole-carbamate-distearoyl lipid/PHSPC/PEG/galactose. As seen Factor VIII is detected in the blood at both time points.

From the foregoing, it can be seen how various objects and features of the invention are met. The lipid of the invention includes a moiety that is responsive to pH such that at a pH of about 7.4 the lipid is close to neutral. Thus, the liposomes when administered to a subject are uncharged, which allows for a longer blood circulation time than that achieved with charged liposomes. Liposomes that are endocytosed or that reach an in vivo region where the pH is lower, for example in a tumor region or in a lysosyme, become charged since the lipid, having a pH responsive moiety becomes positively charged. For example, a lipid having an imidazole moiety, which has a pK of about 6.0, will become predominantly positively charged at pH values less than 6.0. Thus, in an endosome where the pH is between 5-6, the lipid protenates, facilitating uptake and release of the entrapped DNA into the cytoplasm of the cell (Xu and Szoka, Biochemistry, 35:5616-5623 (1996)).

### VI. EXAMPLES

The following examples illustrate but in no way are intended to limit the invention.

Materials

The following materials were obtained from the indicated source: partially hydrogenated soy phosphatidylcholine (Vernon Walden Inc., Green Village, N.J.); cholesterol (Solvay Pharmaceuticals, The Netherlands); dioleoylphosphatidyl ethanolamine (DOPE) and dimethyldioctadecylammonium (DDAB) (Avanti Polar Lipids, Inc., Birmingham, Ala.).

Methods

Dynamic light scattering was performed using a Coulter N4-MD (Coulter, Miami Fla.).

Zeta-Potential: Zeta potential was measured using a ZETASIZER 2000 from Malver Instruments, Inc. (Southborough Mass.). The instrument was operated as follows: number of measurements: 3; delay between measurements: 5 seconds; temperature: 25C; viscosity: 0.89 cP; dielectric constant: 79; cell type: capillary flow; zeta limits: −150 mV to 150 mV.

### Example 1

### Preparation of Exemplary Lipid

A. Preparation of Para-Nitrophenyl Carbonate of Distearoyl Glycerol

As illustrated in FIG. 1, 1,2-disteaeoyl-sn-glycerol (500 mg, 0.8 mmol; Compound I) was dried azeotropically with benzene (3 times with rotary evaporator). Para-nitrophenyl chloroformate (242 mg, 1.2 mmol, 1.5 eq; Compound II), 4-dimethylaminopyridine (10 mg, 0.08 mmol, 0.1 eq), and triethylamine (334 μl, 204 mmol, 3 eq) were added to 1,2-distearoyl glycerol in CHCl₃(5 ml). The reaction mixture was stirred at room temp for 2 h. TLC showed that the reaction was complete. The mixture was diluted with CHCl₃ (50 ml) and extracted with 10% citric acid (3×15 mL). The organic layer was dried (MgSO₄) and evaporated to give a solid. The solid (light orange) was washed with acetonitrile (4×3 mL) to remove excess of p-nitrophenyl chloroformate. The product, para-nitrophenyl carbonate of distearoyl glycerol (Compound II), was dried under vacuum over P₂O₅. Yield: 557 mg (88%). ¹H NMR (360 MHz, DMSO-D6,): δ 0.88 (t, CH₃, 6H); 1.26 (s, CH₂ 58H); 1.62(m, CH₂CH₂CO, 4H); 2.4 (2×t, CH₂CO, 4H); 4.2 (dd, trans CH₂OCO, 1H); 4.35 (m, CH₂OCOO, 2H); 4.5 (dd, cis CH₂OCO, 1H); 5.38 (m, CH₂CHCH₂, 1H); 7.4 (d, C₆H₅, 2H); 8.3 (d, C₆H₅, 2H).

B. Preparation of Carbamate of Histamine and Distearoyl Glycerol

Para-nitrophenyl carbonate of 1,2-distearoyl glycerol (350 mg, 0.44 mmol, Compound II) was added to Histamine (46 mg, 0.40 mmol, 0.9 eq; Compound IV) in CHCl₃ (1 ml) with DMSO (200 μl). Pyridine (300 μl; Compound V) was added to the solution. The reaction mixture was stirred at room temperature overnight (˜20 h). TLC (CHCl₃: MeOH=90:10) showed that the reaction was complete. Solvent was evaporated. The product (Compound VI) was dissolved in CHCl₃, poured on to silica gel (Aldrich, 230-400 mesh, 60 Å) column, and eluted with following solvents, CHCl₃: CH₃COCH₃=90:10, 40 ml (upper spot eluted); CHCl₃: IPA=80:20, 40 ml (product eluted); CHCl₃: IPA=70:30, 40 ml (more product eluted). Fractions containing pure product were combined, and evaporated. The product was dried under vacuo over P₂O₅ and was obtained as white solid (236 mg, 80% yield). ¹H NMR (360 MHZ, CDCl₃/MeOH=1:1 with TMS):δ 0.88(t, CH3, 6H.); 1.28 (s,CH₂,56H; 1.62 (m, CH₂CH₂CO, 4H); 2.34 (2×t, CH₂CO, 4H); 2.77 (t, CH₂CH₂NH, 2H); 3.18 (t, CH₂CH₂CO, 2H); 4.05-4.2 (dd, cis and trans CH₂CHCH₂, 4H); 5.13 (m, CH₂CHCH₂, 1H); 608 (s, Histamine, 1H); 7.53 (s, Histamine, 1H).

### Example 2

### Placebo Liposome Preparation

The lipid (Compound VI) prepared as described in Example 1 and partially hydrogenated soy phosphatidylcholine (PHSPC) in a molar ratio of 40/60 were dissolved in chloroform and/or methanol in a round bottom flask. The solvents were removed by rotary evaporation, and the dried lipid film produced was hydrated with deionized water to produce large multilamellar vesicles.

Comparative liposome formulations were prepared using 100 mole percent PHSPC and with a similar molar ratio of DDAB-cholesterol by a similar methodology.

The liposome size of each formulation was determined by dynamic light scattering.

### Example 3

### Preparation of Liposomes Containing Nucleic Acid

A. Preparation of Liposomes with Entrapped DNA

Complexes were prepared at room temperature as follows. First, 400 μg luciferase reporter plasmid DNA was condensed in 5% glucose solution by slowly adding 100 μg histone with continuous stirring for 10 minutes.

A solution of PHSPC and the pH responsive lipid prepared in Example 1 (Compound VI) in a molar ratio of 40/60 at a total lipid amount of 12,000 nm in 5% glucose was adjusted to pH=4. The condensed DNA solution was added to the acidic liposome solution slowly with continuous stirring for 10 minutes. The final concentration of DNA was 0.25 mg/ml and the total lipid concentration was 7.5 mM. The ratio of DNA total lipids was 1 μg DNA to 30 nmole lipids.

B. DNase I Assay

DNA alone and DNA entrapped in liposomes were treated with DNase I in the presence of 10 mM MgSO₄ at 37⁹C for 30 minutes. After treatment, the liposome/DNase mixture was extracted with phenol/CHCl₃ and CHCl₃ to separate lipids and proteins from DNA. Aliquots of the DNase treated-DNA and of the DNA fraction from the DNase-treated, liposomes having entrapped DNA were loaded onto a 1% agarose gel containing ethidium bromide and electrophoresed to examine the integrity of the DNA. As controls, DNA and liposomes not treated with DNase were loaded onto the gel, along with a 1 Kb DNA standard. The results are shown in FIG. 6.

Although the invention has been described with respect to particular embodiments, it will be apparent to those skilled in the art that various changes and modifications can be made without departing from the invention.

It is claimed:

1. A liposome composition, comprising:
a lipid having the formula 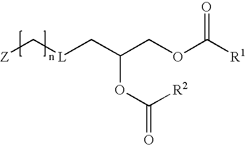
where each of R¹ and R² is an alkyl or alkenyl chain having between 8-24 carbon atoms, and each of R¹ and R² are independently selected;
n=0-20, preferably n=1-20;
L is selected from the group consisting of (i) —X—(C═O)—Y—, (ii) —X—(C═O)—, and (iii) —X—, where X and Y are independently selected from oxygen, NH and a direct bond;
Z is a weakly basic moiety that has a pK of less than 7.4 and greater than about 4.0.

2. The composition of claim 1, wherein X is NH and Y is oxygen.

3. The composition of claim 1, wherein L is a carbamate linkage, an ester linkage or a carbonate linkage.

4. The composition of claim 1, wherein L is NH—(C═O)—O—.

5. The composition of claim 1, wherein Z is an imidazole.

6. The composition of claim 1, comprising between 1-80 mole percent of the lipid.

7. The composition of claim 1, wherein Z is a moiety having a pK value between 5.0-6.5.

8. The composition of claim 1, wherein each of R¹ and R² is an unbranched alkyl or alkenyl chain having between 8-24 carbon atoms.

9. The composition of claim 8, wherein each of R¹ and R² is C₁₇H₃₅.

10. The composition of claim 1, wherein n is between 1-10.

11. The composition of claim 1, further including a therapeutic compound entrapped in the liposomes.

12. The composition of claim 11, wherein the therapeutic agent is a nucleic acid.

13. The composition of claim 12, wherein the nucleic acid is selected from DNA, RNA, fragments thereof and oligonucleotides.

14. The composition of claim 1, further including a ligand for targeting the liposomes to a target site.

15. The composition of claim 14, wherein the ligand is one having binding affinity for endothelial tumor cells and which is internalized by such cells.

16. The composition of claim 15, wherein the ligand is selected from the group consisting of E-selectin, Her-2 and FGF.

17. The composition of claim 1, wherein said liposomes further include between 5-20 mole percent of a vesicle-forming lipid derivatized with a hydrophilic polymer chain.

18. The composition of claim 17, wherein said hydrophilic polymer chain is polyethyleneglycol.

19. A lipid having the formula: 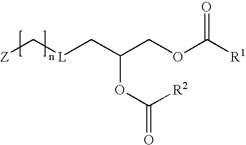
where each of R¹ and R² is an alkyl or alkenyl chain having between 8-24 carbon atoms, and each of R¹ and R² are independently selected;
n=0-20, preferably n=1-20;
L is selected from the group consisting of (i) —X—(C═O)—Y—, (ii) —X—(C═O)—, and (iii) —X—, where X and Y are independently selected from oxygen, NH and a direct bond;
Z is a weakly basic moiety that has a pK of less than 7.4 and greater than about 4.0.

20. The lipid of claim 19, wherein X is NH and Y is oxygen.

21. The lipid of claim 19, wherein L is a carbamate linkage, an ester linkage or a carbonate linkage.

22. The lipid of claim 19, wherein L is NH—(C═O)—O—.

23. The lipid of claim 22, wherein Z is an imidazole.

24. The lipid of claim 19, wherein Z is a moiety having a pK value between 5.0-6.5.

25. The lipid of claim 19, wherein each of R¹ and R² is an unbranched alkyl or alkenyl chain having between 8-24 carbon atoms.

26. The lipid of claim 23, wherein each of R¹ and R² is C₁₇H₃₅.

27. The lipid of claim 19, wherein n is between 1-10.

28. A liposome comprising the lipid according to claim 19.

29. A liposome comprising the lipid according to claim 26.

30. A method for delivery of a therapeutic agent to the cytoplasm of a cell, comprising
preparing liposomes comprising a lipid having the formula 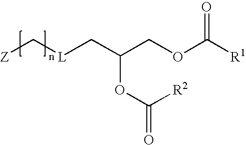
where each of R¹ and R² is an alkyl or alkenyl chain having between 8-24 carbon atoms, and each of R¹ and R² are independently selected;
n=0-20, preferably n=1-20;
L is selected from the group consisting of (i) —X—(C═O)—Y—, (ii) —X—(C═O)—, and (iii) —X—, where X and Y are independently selected from oxygen, NH and a direct bond;
Z is a weakly basic moiety that has a pK of less than 7.4 and greater than about 4.0; and
administering the liposomes to a subject.

31. The method of claim 30, wherein said preparing includes entrapping in the liposomes a nucleic acid.

32. The method of claim 31, wherein the nucleic acid is an oligonucleotide.

33. The method of claim 30, wherein said preparing includes entrapping in the liposomes a protein.
